(19) United States
(12) Patent Application Publication
(10) Pub. No.: US 2023/0420095 A1
(43) Pub. Date: Dec. 28, 2023

(54) PROVIDING SUPPLEMENTAL INFORMATION FOR A PATIENT REPORT TO PRODUCE AN UPDATED PATIENT REPORT PROCESSED TO DETERMINE MEDICAL FINDINGS AND A MEDICAL BEST PRACTICE RECOMMENDATION

(71) Applicant: RADIOLOGY PARTNERS, INC., El Segundo, CA (US)

(72) Inventors: Nina KOTTLER, San Diego, CA (US); Kelly DENNEY, Hilliard, OH (US); Jai SALZWEDEL, Columbus, OH (US); Kent HUTSON, Palmer Lake, CO (US); Tuguldur SUKHBOLD, Dublin, OH (US); Collin MCCABE, Westerville, OH (US); Telford BERKEY, London, OH (US)

(21) Appl. No.: 18/317,007

(22) Filed: May 12, 2023

Related U.S. Application Data
(60) Provisional application No. 63/367,211, filed on Jun. 28, 2022.

(51) Int. Cl. G16H 15/00; G16H 50/20; G16H 10/60

(52) U.S. Cl. CPC G16H 15/00; G16H 50/20; G16H 10/60

(57) ABSTRACT

Provided are a computer program product, system, and method for providing supplemental information for a patient report to produce an updated patient report processed to determine medical findings and a medical best practice recommendation. A patient report is processed to determine supplemental information and transmit to the user interface to render. A first updated patient report is received including content for the supplemental information. A classifier program processes the first updated patient report including the content for the supplemental information to classify into a medical finding. A determination is made of a medical best practice recommendation associated with the medical finding. The medical finding and the medical best practice recommendation are transmitted to the user interface to render. A second updated patient report is received including at least one of the medical finding and the medical best practice recommendation the user selected to include.

### CROSS-REFERENCE TO RELATED APPLICATION

This application claims the benefit of U.S. Provisional Application No. 63/367,211, filed Jun. 28, 2022, which provisional application is incorporated herein by reference in its entirety.

### BACKGROUND OF THE INVENTION

### 1. Field of the Invention

The present invention relates to a computer program product, system, and method for providing supplemental information for a patient report to produce an updated patient report processed to determine medical findings and a medical best practice recommendation.

### 2. Description of the Related Art

Medical software, deploying machine learning and artificial intelligence algorithms, is used to assist medical professionals with preparing patient reports, including assisting with billing and reimbursement, and recommending treatments and courses of action. Medical software utilizing machine learning and artificial intelligence is also provided to assist radiologists in preparing patient reports concerning imaging examinations. Machine learning has also been used to improve medical descriptions to assist with billing and insurance reimbursements.

There is a need in the art for improved techniques for providing a human user interface to enable users, such as doctors and medical personnel, to process and edit patient reports to have optimal content.

### SUMMARY

Provided are a computer program product, system, and method for providing supplemental information for a patient report to produce an updated patient report processed to determine medical findings and a medical best practice recommendation. A patient report is processed to determine supplemental information for the patient report. The patient report has information on a patient encounter at a medical clinic. The supplemental information is transmitted to the user interface to render in the user interface. A first updated patient report is received including content for the supplemental information a user of the user interface selected to insert to the patient report. A classifier program processes the first updated patient report including the content for the supplemental information to classify into a medical finding based on content in the patient report and the content for the supplemental information. A determination is made of a medical best practice recommendation associated with the medical finding outputted from the classifier program. The medical finding and the medical best practice recommendation are transmitted to the user interface to render in the user interface. A second updated patient report is received including at least one of the medical finding and the medical best practice recommendation the user selected to include into the patient report.

### BRIEF DESCRIPTION OF THE DRAWINGS

FIG. 1 illustrates an embodiment of a computing environment.

FIGS. 2a, 2b, 2c, 2d, 2e, 2f, and 2g illustrate examples of components of a report user interface to render and allow for interaction with a patient report.

FIG. 3 illustrates an embodiment of user report settings to control content added to a patient report in the report user interface.

FIG. 4 illustrates an embodiment of operations to generate and process a patient report.

FIG. 5 illustrates an embodiment of operations to process an updated patient report.

FIG. 6 illustrates an embodiment of operations to process a patient report to generate a medical finding and best practice recommendation.

FIG. 7 illustrates an embodiment of operations for a user interface to enable a user to insert a medical finding and best practice recommendation into a patient report.

FIG. 8 illustrates an embodiment of a supplemental information tracking instance to track a supplemental information alert transmitted to the report user interface.

FIG. 9 illustrates an embodiment of operations to invoke services to process a patient report to determine the supplemental information for the patent report.

FIG. 10 illustrates an embodiment of operations for a user interface to render a supplemental information alert and enable a user to insert content for the supplemental information into the patient report.

FIG. 11 illustrates an embodiment of operations performed by a service to standardize inhomogeneous data in the patient report.

FIG. 12 illustrates an embodiment of operations performed by a service to determine discrepancies with patient metadata in a patient report.

FIG. 13 illustrates an embodiment of operations performed by a service to determine missing sections in a patient report.

FIG. 14 illustrates an embodiment of an attestation service.

FIG. 15 illustrates an embodiment of operations performed by the attestation service to determine attestations to provide for a patient report.

FIG. 16 illustrates an embodiment of operations performed to retrain a classifier program and rules engine based on receiving a rejection of a medical finding or best practice recommendation.

FIG. 17 illustrates a computing environment in which the components of FIG. 1 may be implemented

### DETAILED DESCRIPTION

Described embodiments provide improvements to computer technology to control how a user interface provides information in real-time on a patient encounter that is used to determine medical findings and best practice recommendations to include in a patient report on the patient encounter. Described embodiments further provide improvements for controlling how information on the medical findings and best practice recommendations are added to the patient report. For instance, a doctor or clinician may enter observations and a diagnosis of a patient condition in a patient report rendered in a report user interface, such as by observing the patient, lab test results, and medical images. The clinician or doctor may want to be immediately informed of the relevant best practices based on the observed findings. The clinician or doctor may also have preferred settings on how presented information on medical findings and best practice recommendations are inserted into the report. With described embodiments, user settings are used to control how real-time information presented in an alert in a report user interface is inserted into the patient report.

Further embodiments provide improvements to computer technology for determining whether a patient report having information on a patient encounter includes attestations having explanatory information on the patient encounter. With described embodiments, immediate information on patient reports lacking sufficient attestations is rendered in the report user interface to enable the clinician or doctor preparing the report to insert the needed attestations. Further, the updated patient report including the inserted attestations may be further processed to provide further refined medical findings and best practice recommendations to further add to the patient report.

Yet further embodiments provide improvements to computer technology to determine supplemental information to add to a patient report that is rendered in a report user interface to enable a user to insert content for the supplemental information into the patient report. With the described embodiments, the updated patient report with the content on the supplemental information may be provided to a classifier program to determine a medical finding and best practice recommendation based on content in the updated patient report including the content added for the supplemental information. This allows for adjustments to the medical finding and best practice recommendation based on the recently inserted content for the supplemental information.

The described embodiments alert the user with changes to the medical findings and best practice recommendations resulting from changes entered into the patient report via the user interface to provide real-time feedback on such changes.

Described embodiments provide improvements to the computer technology for determining real-time entry or changes to inputted content, such as user entered observations and classifications, to forward to a program, such as a machine learning classifier program and rules engine, to provide real-time feedback on the entered changes. In described embodiments, when changes are made to the patient report, the updated patient report may be subject to further processing by the classifier program and rules engine to determine updated medical findings and best practice recommendations, as well as supplemental information for the patient report.

In certain embodiments, the user entered observations may apply to processing user entered medical observations of patient data, observation of the patient conditions and attributes, digital images, and physical samples, such as biopsies and bodily fluid samples, to provide real-time feedback of best practices and recommendations to the user entered medical observations. Upon determining changes in the user entered findings, such as observations and classifications, the new inputted observations may be sent to the classifier program to determine from the user entered medical observations a predefined machine classification, such as a clinical diagnosis or recognized condition, to provide to a rules engine. The rules engine determines medical best practices based on applying a series of rules from the rules engine to the machine classification. The best practices and/or reference material, recommendations, calculations, summary of information from other sources, actions and other relevant information may then be immediately returned to render in the user interface to provide immediate feedback, such as for best practices and changes to best practices for user entered observations.

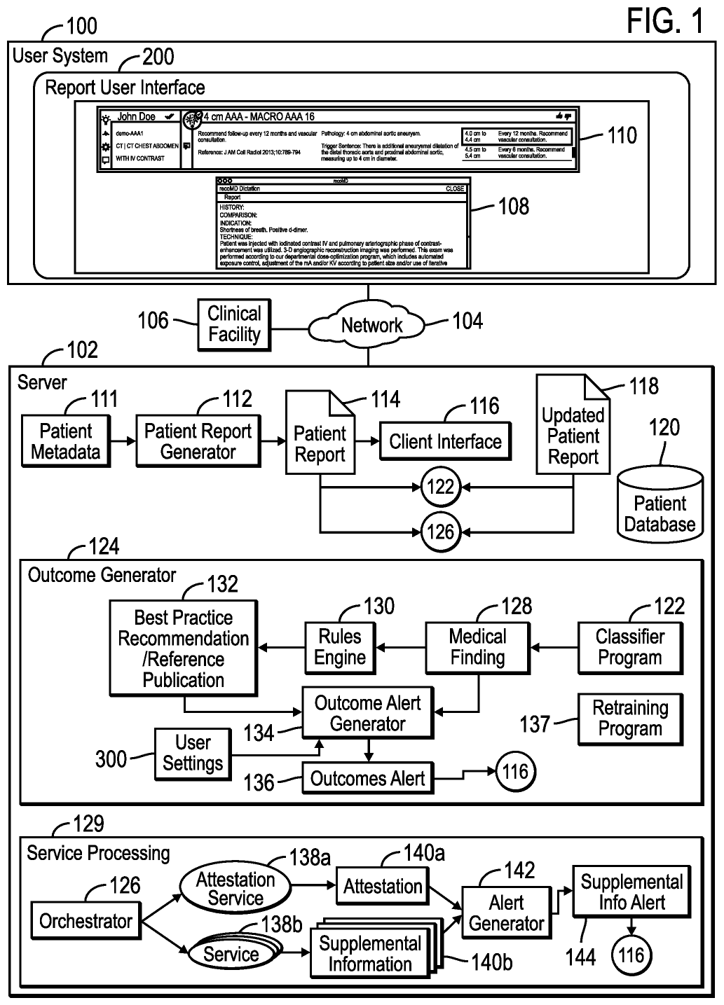

FIG. 1 illustrates an embodiment of a computing environment in which embodiments are implemented. A user system 100 is in communication with a server 102 over a network 104. The server 102 may receive information on a patient encounter, including imaging, from a clinical facility 106 having the machines to perform the imaging. The user system 100 includes a report user interface 200 for generating a patient report 108 and various alerts 110 received from the server 102 over the network 104. The patient report 108 includes information on a patient encounter, including information an examination of the patient and medical imaging results, including Magnetic Resonance Imaging (MRI), X-Rays, ultra sound, etc. The report user interface 200 may continually render the patient report 108 and alerts 110. User changes to the data in the patient report 108 are immediately forwarded to the server 107 to provide further medical findings and best practice recommendations.

The server 102 receives electronic patient metadata 111 from a patient facility system 106, which may be in the Digital Imaging and Communications in Medicine (DICOM) format. The patient metadata 111 may include a digital captured image and information such as patient information, e.g., ID, sex, age, etc., type of procedure, e.g., equipment type to capture image, product description, procedure code, and procedure order, e.g., referring physician, reason for exam, etc. Additional information may include information about how the image was acquired, radiation doses, etc. The device used to capture the digital image may comprise include, CT (computed tomography), MRI (magnetic resonance imaging), ultrasound, X-ray, fluoroscopy, angiography, mammography, breast tomosynthesis, PET (positron emission tomography), SPECT (single-photon emission computed tomography), Endoscopy, microscopy, whole slide imaging, OCT (optical coherence tomography). etc.

A patient report generator 112 processes the patient metadata 111 to include in a patient report 114. The first time the patient report 114 is generated from the patient metadata 111 it is provided to a client interface 116 to transmit to the user interface 200 at the user system 100 to render as patient report 108. The user interface 200 further transmits user updates to the patient report 108 to the server 102 in the form of updated patient reports 118. Information on a patient encounter for a patient and copies of the patient report 114 and updated patent report 118 versions may be stored associated with the patient in a patient database 120.

The initial patient report 114 and the updated patient report 118 are provided to a classifier program 122 in an outcome generator 124 to initiate determining a medical finding and best practice recommendation from the patient report 114, 118. The reports 114 and 118 are further provided to an orchestrator 126 in service processing 129 to invoke services to process the patient reports 114, 118 to determine supplemental information to present in alert panels 110 in the report user interface 200.

The outcome generator 124 includes components to process the reports 114, 118 to determine a medical finding 128, e.g., the pathology or diagnosis, based on content in the patient reports 114, 118. The classifier program 122, which may comprise a machine learning program, processes information in the patient reports 114, 118, such as radiologist entered findings and information, and generate a medical finding 128. The medical finding 128 is forwarded to a rules engine 130 that uses a decision tree or table that associates/maps specific medical best practice recommendations (“BPRs”) 132 with medical findings 128 outputted from the classifier program 122. For instance, if the classifier program 122 detects a clinical diagnosis, then the rules engine 130 may determine the BPR 132 to treat a medical finding 128, e.g., clinical diagnosis, outputted from the classifier program 122, such as a drug therapy, surgical treatment, further testing, further follow-up visit, etc. In this way, the rules engine 130 may provide a best practices recommendation 132 for each possible classified medical finding 128 outputted from the classifier program 122.

In one embodiment, the medical findings 128 may comprise a size of an observed condition on a patient, such as a size of an abdominal aortic aneurysms (AAA), observed features, such as size, shape, etc., of an incidental thyroid nodule, ovarian cyst, non-incidental thyroid nodule, enlarged thyroid, simple ovarian cyst, etc. The rules engine 130 may specify particular best practice recommendations 132 given different sizes of the AAA, such as recommended follow-ups after so many years or a follow-up and an additional vascular consultation. In certain embodiments, the updated patient report 118 is provided to the classifier program 122 in response to the radiologist adding text in the patient report 108 rendered in the report user interface 200.

An advantage of having the rules engine 130 separate from the classifier program 122 is that the rules engine 130 may be independently updated to provide new results, or best practices for the classified medical finding 128 outputted from the classifier program 122.

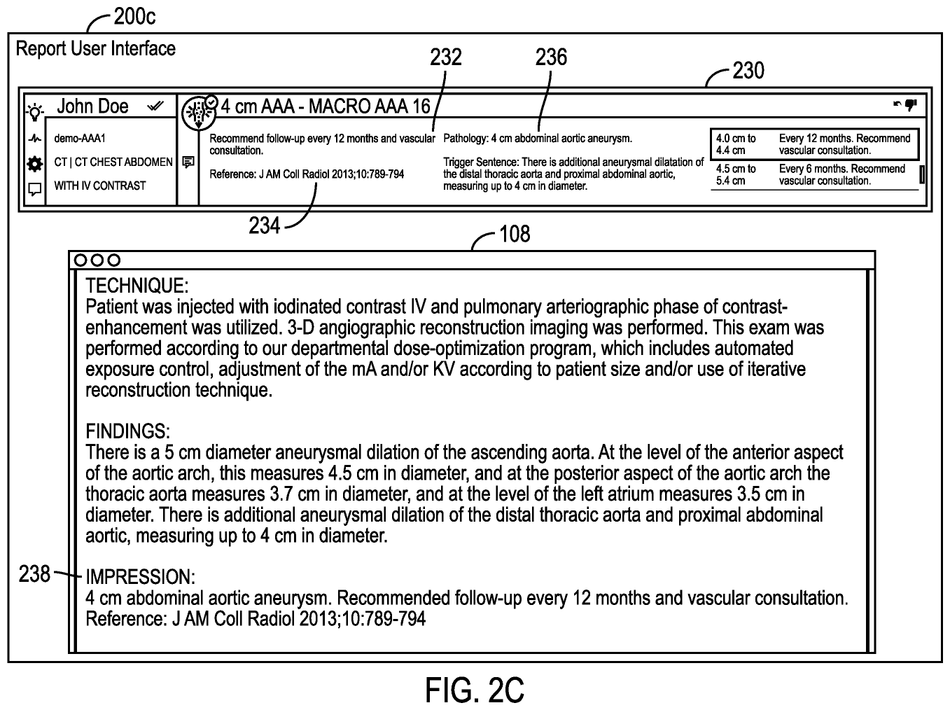

The medical finding 128, BPR 132 and a reference to a publication indicating the outputted BPR 132 for the medical finding 128 are forwarded to an outcome alert generator 134 which utilizes user settings 300 (FIG. 3) to generate an outcomes alert 136 to forward to the client interface 116 to transmit to the report user interface 200 at the user system 100. The outcomes alert 136 may indicate the medical finding 128, best practice recommendation 132, and a reference to the publication, and the user settings 300 may provide code to control the outcomes 128 and 132 inserted from the alert 140 into the patient report 108.

The outcome generator 124 may further include a retraining program 137 to retrain the machine learning implementation of the classifier program 122 based on feedback of the output of the medical findings 128 and to update the rules engine 130 to provide updated or preferred medical best practice recommendations for determined medical findings.

The service processing 129 includes components to scan the patient report 114, 118 to determine supplemental information indicating processing of the report to improve the predictability of the information in the patient report provided to the classifier program 122 to generate the appropriate medical finding 128. The orchestrator 126 may invoke multiple services 138a, 138b to perform different processing of the received patient report 114, 118 to determine supplemental information 140a, 140b for the report. For instance, an attestation service 138a may perform natural language processing (NLP) or look for predefined words indicating a patient examination and then output supplemental information in the form of an attestation 140a, or explanatory description of the identified patient examination, that should be inserted into the patient report 114, 118. This added attestation 140a may include descriptions that are known to facilitate reimbursement for the patient encounter. Other services 138b may process the patient report 114, 118 to search for discrepancies with the report, such as inhomogeneous data resulting from variances in terminology used at different clinical facilities 106, missing sections needed to improve the classification by the classifier program 122 and facilitate reimbursement, patient information missing or having discrepancies, etc. The output 140b of the services 138b may comprise supplemental information 140b describing the discrepancies with the patient report 114, 118. The output attestation 140a and supplemental information 140b are provided to an alert generator 142 to generate a supplement information alert 144 including the supplemental information 140a, 140b that when rendered in the report user interface 200 enables the user to insert content for the supplemental information 140a, 14b into the patient report 108.

In certain embodiments, the classifier program 122 and services 138a, 138b may use machine learning and deep learning algorithms, such as decision tree learning, association rule learning, neural network, inductive programming logic, support vector machines, Bayesian network, etc. For artificial neural network program implementations, the neural network may be trained using backward propagation to adjust weights and biases at nodes in a hidden layer to produce the classification, such as a medical finding 128. In backward propagation used to train a neural network machine learning module, biases at nodes in the hidden layer are adjusted accordingly to produce the medical finding having specified confidence levels based on the input patient report 114, 118. Backward propagation may comprise an algorithm for supervised learning of artificial neural networks using gradient descent. Given an artificial neural network and an error function, the method may calculate the gradient of the error function with respect to the neural network's weights and biases.

In one embodiment, the classifier program 122 may comprise a machine learning program that is trained using a training set comprising previously generated patient reports that have been classified with a ground truth classification, and the classifier program 122 is trained to produce the ground truth classifications provided for the training set of reports. For instance, if the training set comprises results of a radiologist entering observations from an MRI reading, then the provided ground truths would be radiologist determined classifications or clinical diagnosis based on those findings. The classifier program 122 would then be trained with those findings to produce the medical findings and clinical diagnosis assigned to those findings and observations.

In an alternative embodiment, the classifier program 122 and services 138a, 138b may be implemented not as a machine learning module, but implemented using a rules based system to determine the outputs from the inputs. The classifier program 122 and services 138a, 138b may further be implemented using an unsupervised machine learning module, or machine learning implemented in methods other than neural networks, such as multivariable linear regression models.

The arrows shown in FIG. 1 between the components and objects in a memory of the server 102 represent a data flow between the components.

The network 104 may comprise one or more networks including Local Area Networks (LAN), Storage Area Networks (SAN), Wide Area Network (WAN), peer-to-peer network, wireless network, the Internet, etc.

Generally, program modules, such as the program components 112, 116, 122, 126, 130, 134, 137, 138a, 138b, 142, 200, 200a . . . 200g, 1404 may comprise routines, programs, objects, components, logic, data structures, and so on that perform particular tasks or implement particular abstract data types. The program components and hardware devices of the computing device 100 and server 102 of FIG. 1 may be implemented in one or more computer systems, where if they are implemented in multiple computer systems, then the computer systems may communicate over a network.

The program components 112, 116, 122, 126, 130, 134, 137, 138a, 138b, 142, 200, 200a . . . 200g, 1404 may be accessed by a processor from memory to execute. Alternatively, some or all of the program components 112, 116, 122, 126, 130, 134, 137, 138a, 138b, 142, 200, 200a . . . 200g, 1404 may be implemented in separate hardware devices, such as Application Specific Integrated Circuit (ASIC) hardware devices.

The functions described as performed by the programs 112, 116, 122, 126, 130, 134, 137, 138a, 138b, 142, 200, 200a . . . 200g, 1404 may be implemented as program code in fewer program modules than shown or implemented as program code throughout a greater number of program modules than shown.

Some or all of the components shown as implemented in the server 102, such as the programs 112, 116, 122, 126, 130, 134, 137, 138a, 138b, 142, 1404, may be implemented in the user system 100 or other computing systems in the network 104. In certain embodiments, the server 102 may comprise a cloud server providing cloud services for the outcome generator 124 and the service processing 129. The server 102 may also provide medical findings 128, best practice recommendations 132, attestations 140a, and supplemental information 140b to user systems 100 at different medical facilities, locations, hospitals, etc., to provide cloud based services.

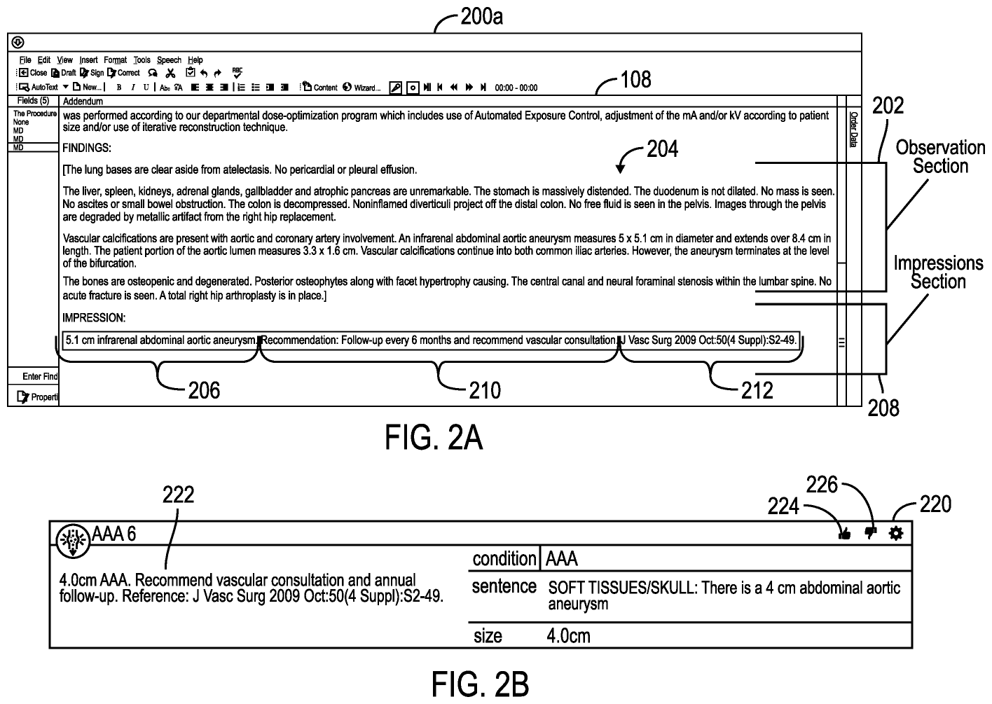

FIGS. 2a, 2b, 2c, 2d, 2e, 2f, 2g provide implementations of the report user interface 200. FIG. 2a illustrates an embodiment of a user interface 200a rendering a patient report 108 in which a user may input data, and includes an observations section 202 in which a radiologist or other user may enter medical observations 204 about an entity being diagnosed, such as a person or animal. The medical observations may be described from an image or video, direct observation of a phenomena, etc. The user may further enter a medical finding or diagnosis 206 in the impression section 208, or any other section, providing a classification of the observations of the patient or patient test results, e.g., medical imaging, blood tests, etc. The impressions section 208 may further render a best practice recommendation 210 and reference to a publication 212 providing support for the best practice recommendation 210 produced by the rules engine 130. The classifier program 122 may receive the user entered observations 202, and information in any fields of the patient report 108, including any user classifications and findings, and information from patient metadata 111, and classify into a medical finding 128, such as a clinical diagnosis, category, etc., which is then provided to the rules engine 130 to determine a best practice recommendation or course of action to render in field 210 in the user interface 200a concerning the machine classified findings.

The patient report 108 may include additional sections, such as a history section, such as shown in FIG. 2d, providing history of a patient, an indication section indicating a pathology, a technique section describing a technique of an examination, such as information on machine used for examination, settings for machine, e.g., MRI, X-Ray, CT Scan, etc., and a comparison section comparing current patient state to a previous diagnosis or test, such as previous imaging. Other of the FIGS. 2b, 2c, 2d, 2e, 2f show the different sections of the patient report 108. The patient report generator 112 may initially populate information in the sections of the patient report 108 with patient metadata 111.

Although in FIG. 2a, sections are labeled as “Findings” and “Impressions” the content described as being rendered in these sections may be in any sections having any name or in a combined section with or without a name. Further, the user of the user interface 200a may change the section names in the patient report 108.

In certain embodiments, the report user interface 200 may be continually displayed rendered and available to receive user entry of data into any section of the patient report 108. Entry of data or certain predefined words may cause the patient report 108 to be forwarded to the outcome generator 124 to determine a medical finding 128 and medical best practice recommendation 132 and forwarded to the service processing 129 for further service processing of the updated information.

FIG. 2b illustrates an example of an alert panel 220 rendered in the report user interface 200a, which may be displayed as a dialog box or pop-up alert, that alerts the radiologist or user of a medical best practice recommendation 222 or other information based on the user entered medical observations 204 and 206. The user may select an accept selection control 224 to cause the best practice recommendation 222 to be entered into field 210 of the user interface 200 to accept the machine generated best practices from the rules engine 130. Further, the medical finding 222 rendered in the panel 220, which may comprise the medical finding 128 from the classifier program 122, may be inserted into field 206 of the patient report 108. The user may also select a reject selection control 226 to reject the best practices recommendation and medical finding. The user may control the alert panel 220 to insert the medical finding, best practice recommendation, and reference to publication describing the best practice recommendation 222 into the patient report 108 by voice command, copy and paste from the field 222 in the alert user interface 220 into the best practices field 210 of the user interface 200, or click a button, such as 224 to cause the automatic insertion of the best practices recommendation in the field 222 of the alert user interface 220 into the user interface 200, such as into field 210.

FIG. 2c illustrates an embodiment of the report user interface 200c displaying the patient report 108 and an alert panel 230 to render a medical best practice recommendation 232, comprising the best practice recommendation 132 produced from the rules engine 130, a reference to a publication 234, and a medical finding 236, comprising the medical finding 128 produced by the classifier program 122. The information 232, 234, 236 is inserted into the patient report 108 in the impressions section 238, or any other section.

FIG. 2d illustrates an embodiment of the report user interface 200d displaying the patient report 108 and two alert panels, one a missing history alert panel 240 generated by a service 138b that detects a missing history section from the patient report 108 and an attestation panel 242 generated by the attestation service 138a providing an attestation of a radiation dose used in the imaging that is described in the “Technique” section of the patient report 108. Including this attestation of the radiation dosage may facilitate higher levels of reimbursement. If the user enters a history section into the patient report 108, then the alert 240 may disappear. The user may select or click the attestation panel 242 to cause the radiation dose attestation displayed in the panel 242 to be entered into the patient report 108.

FIG. 2e illustrates an embodiment of the report user interface 200e displaying the patient report 108 and alert panels 240, 242. The patient report 108 shows the history section missing, which may be added above the comparison section at the beginning of the report 108.

FIG. 2f illustrates an embodiment of the report user interface 200f displaying the patient report 108 with the history alert panel 240 from FIG. 2e removed because the user added the history section 246 in the patient report 108. The attestation panel 242 remains if the attestation described in the panel 242 has not yet been added to the patient report 108.

FIG. 2g illustrates an embodiment of the report user interface 200g displaying an alert panel 230, the patient report 108, and a user settings panel 250 that includes settings in the recommendation output 252 to allow the user to specify what information of the medical findings and best practice recommendation 254 to automatically insert from the alert panel 230 into the patient report 108, including full (medical finding, best practice recommendation, and reference) 256, only recommendation 258, or recommendation and reference to publication 260.

The user interface 200, 200a, 200c . . . 200g provides improvements to computer technology for rendering medical findings and best practice recommendations, through an alert panel, that are generated from a machine learning classifier program 122 and rules engine 130. Further described embodiments provide improved techniques for generating alerts from supplemental information generated by services that process the patient report to determine whether to separately render information on supplemental information and further changes to make to the report. Described embodiments further allow immediate computation of new medical findings 128, best practice recommendations 132, and supplemental information 140a, 140b to render in the user interface 200 in response to user changes to the patient report to provide immediate real time display of new outcomes and supplemental information in the report user interface 200.

FIG. 3 illustrates an embodiment of user settings 300 the user may modify through the user settings panel 250 rendered in the report user interface 200g (FIG. 2g). In the user settings 300, the user may select through indicators 304, 306, 308 the information from the alert panel 230 rendered in the user interface 200c (FIG. 2c) that will be inserted into the patient report 108, such that the user may indicate in the user settings 250 to have the report user interface 200g insert medical findings 304, best practice recommendations 306, and/or reference to publication 308 from the corresponding information 236, 232, 234 rendered in the alert panel 230. The user 302 of the user settings 300 may identify a particular radiologist, to allow different radiologists and clinicians to save preferences for what alert panel 230 information 232, 234, 236 is automatically inserted into the patient report 108.

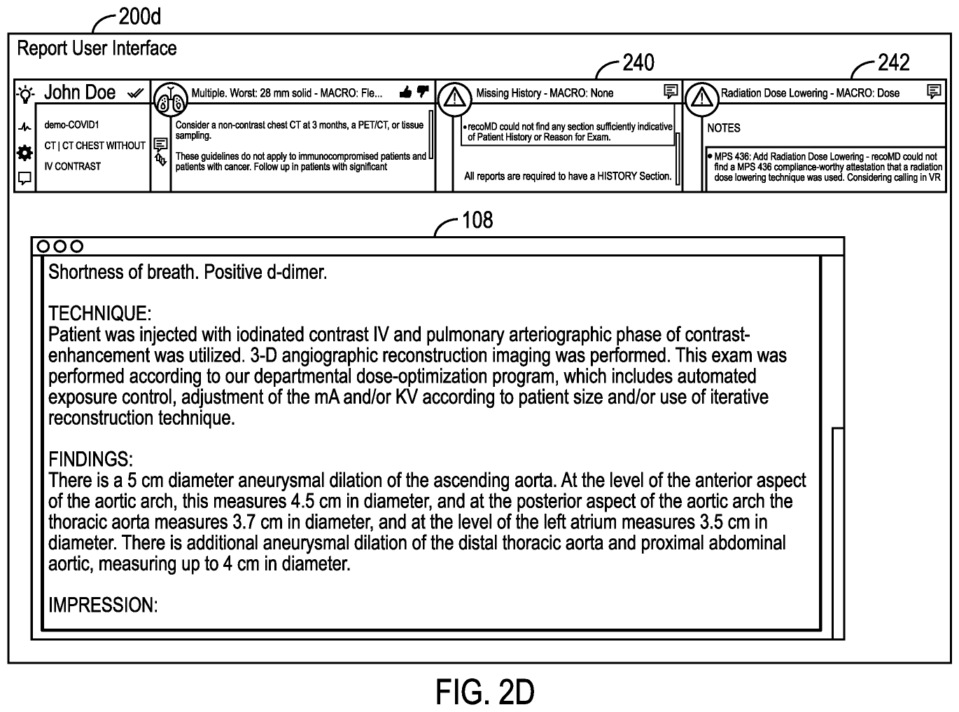

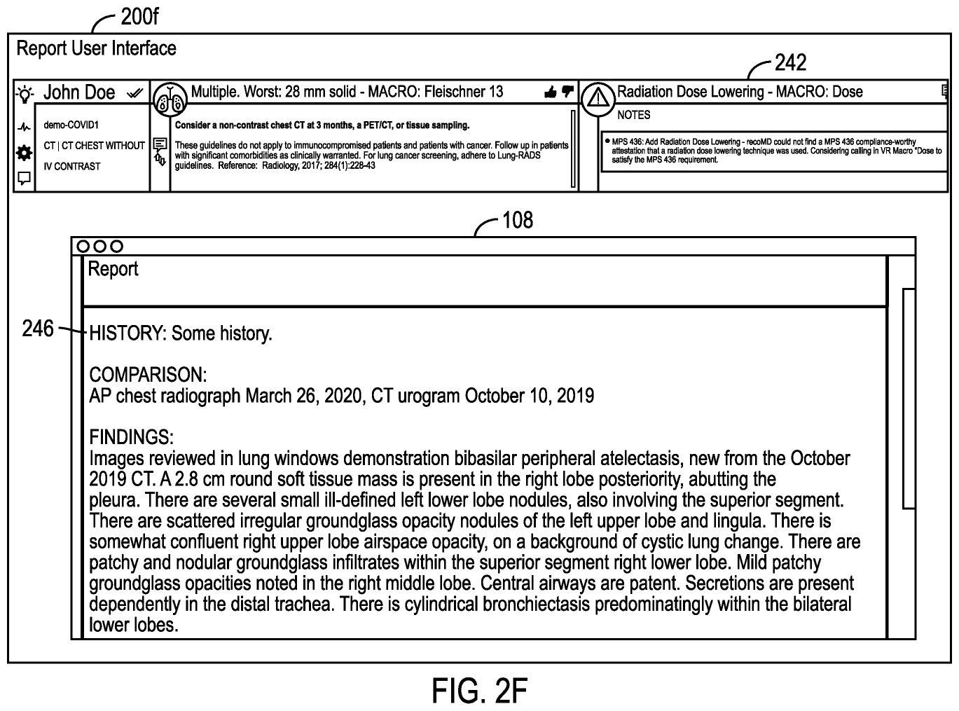

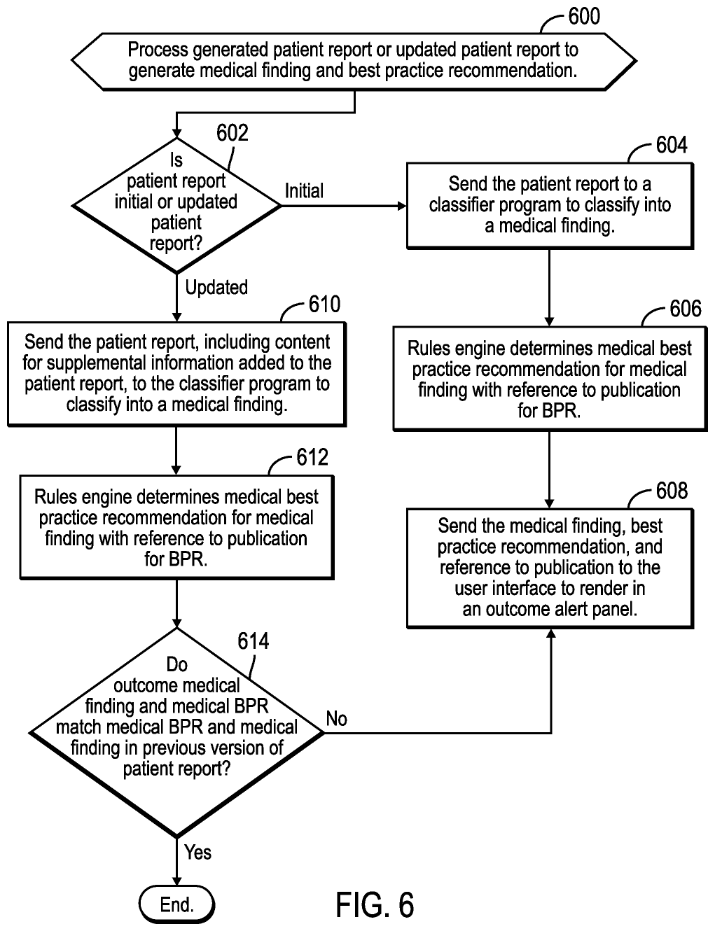

FIG. 4 illustrates an embodiment of operations performed by the patient report generator 112 in the server 102 to generate an initial patient report 114. Upon initiating (at block 400) operations to generate the patient report 114 for a patient, the patient report generator 112 processes (at block 402) patient metadata 111, e.g., DICOM data, from a clinical facility 106 at which the patient was examined, such as using an imaging machine such as an MRI, CT Scan, etc., and populates (at block 404) fields of the patient report 114 with the patient metadata 111 for the patient. The generated patient report 114 is transmitted (at block 406) to the user system 100 to render in the report user interface 200. The generated patient report 114 is further sent (at block 410) to the outcome generator 124 to send to the classifier program 122 to process according to FIG. 6. The patient report 114 is further sent (at block 410) to the service processing 129 to invoke services 138a, 138b, either in parallel or serially, to process the patient report 114 to determine supplemental information 140a, 140b for alerts according to FIG. 9, 11, 12, 13, 17. The patient report 114 is saved (at block 412) in the patient database 120 for the patient.

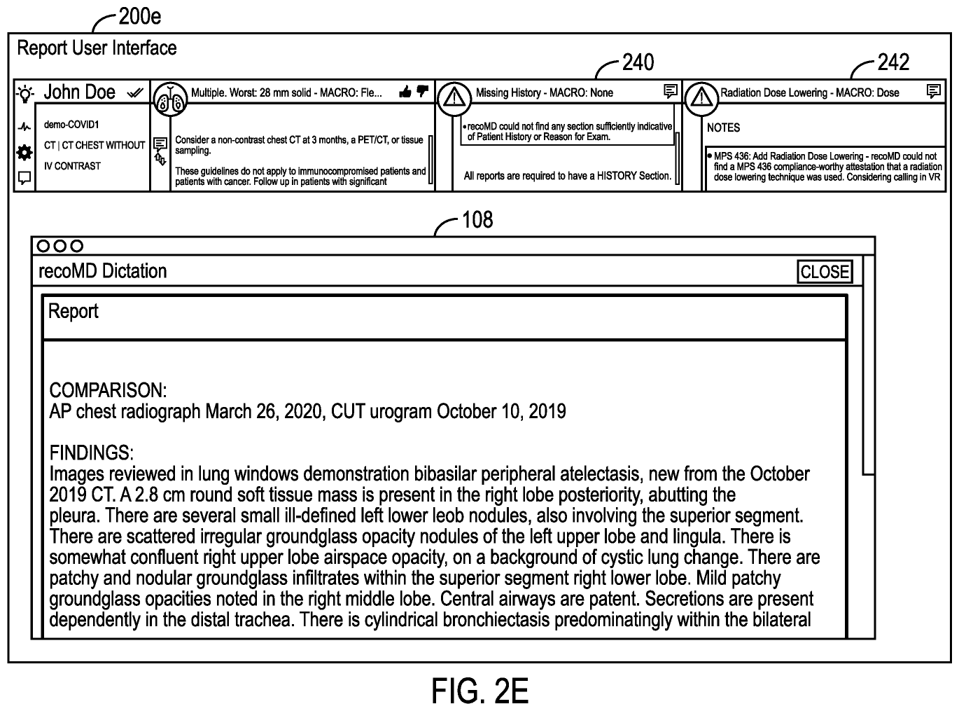

FIG. 5 illustrates an embodiment of operations performed by the client interface 116 upon receiving an updated patient report 118 from the report user interface 200. Upon receiving the updated patient report 118, the operations at blocks 408, 410, and 412 of FIG. 4 are performed with respect to the updated patient report 118 to determine updated medical findings and best practice recommendations, and supplemental information for the updated patient report 118.

FIG. 6 illustrates an embodiment of operations performed by the outcome generator 124 components to determine medical findings 128 and best practice recommendations 132 for the initial patient report 114 and the updated patient report 118 having updates added by the user of the user system 100. Upon processing (at block 600) the initial 114 or updated 118 patient report, if the patient report is the initial patient report 114, then the patient report generator 112 sends (at block 604) the patient report 114 to the classifier program 122 to classify into a medical finding 128. The rules engine 130 determines (at block 606) the medical best practice recommendation 132 for the medical finding 128 with reference to the publication for the BPR. The medic al finding 128 and best practice recommendation 132 and reference to publication are sent (at block 608) by the client interface 116 to the report user interface 200 to render in an outcome alert panel 230, such as in FIG. 2c.

If (at block 602) the patient report is an updated patient report 118, sent by the report user interface 200, the patient report including any content for supplement information added by the user is sent (at block 610) to the classifier program 122 to determine the medical finding 128. The medical finding 128 is forwarded (at block 612) to the rules engine 130 to determine the medical best practice recommendation 132 and reference to a publication referencing the BPR. If (at block 614) the determined medical finding 128 and best practice recommendation 132 do not match those from the immediate previous version of the patient report, then control proceeds to block 608 to send the determined medical finding 128 and best practice recommendation 132 to the report user interface 200 to render. Otherwise, if (at block 614) they match, meaning no change to a previously sent medical finding 128 and BPR 132, control ends without sending the unchanged medical finding and BPR to the report user interface.

With the embodiment of FIG. 6, the initial patient report and the immediately updated patient report are sent to the outcome generator 124 to determine medical findings 128 and a BPR 132 and whether they should be sent to the report user interface 200 to allow insertion or updating a previously inserted medical finding and BPR. User entered findings, such as observations and/or impressions, entered in real time are sent to the classifier program 122 and the rules engine 130 to calculate immediate medical findings 128 and medical best practice recommendations 132 to return to the outcome alert generator 134 to generate an outcomes alert 136 to send to immediately render the results in the report user interface 200c in the alert panel 230. This immediate feedback to an updated patient report 118 enables the user to see in real-time the changes in the results, actions or best practices resulting from entering new findings, and determine whether to have them entered into the patient report being generated in the report user interface 200.

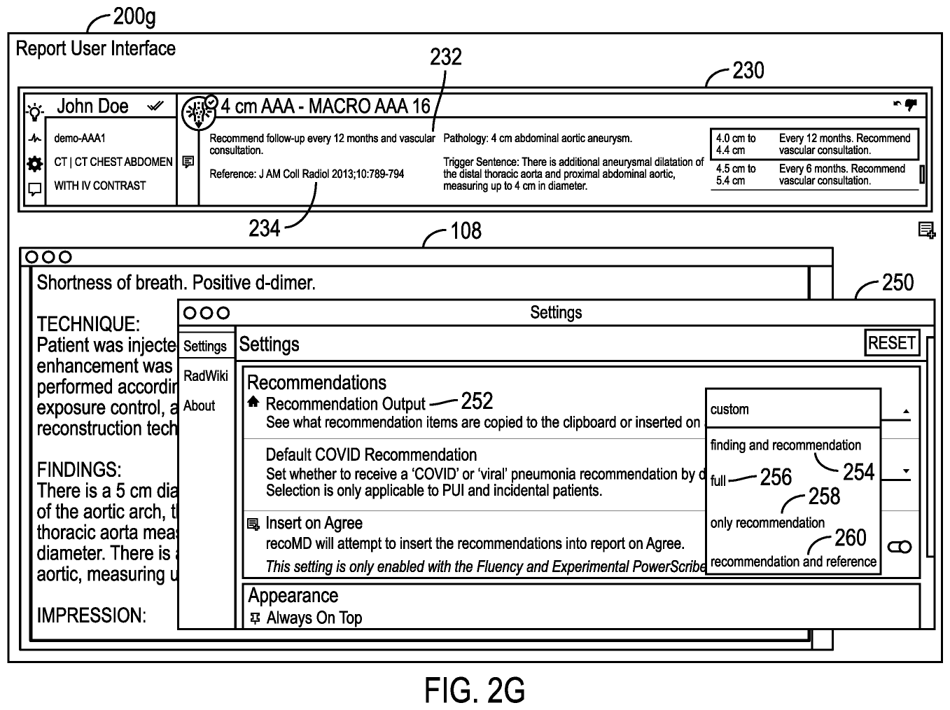

FIG. 7 illustrates an embodiment of operations performed by the report user interface 200c to render an alert panel 230 (FIG. 3) for received medical finding 128, best practice recommendation (BPR) 132, and reference to publication providing BPR. Upon receiving (at block 700) from the server 102 a received medical finding 128, best practice recommendation (BPR) 132, and reference to publication providing BPR, the report user interface 200c (FIG. 2c) displays the medical finding 236, BPR 232, and reference to publication 234 in the alert panel 230 as shown in FIG. 2c. Upon receiving (at block 704) user selection to add information from the alert panel 230 to the patient report 108, if (at block 706) the user settings 300 indicate in indicator 304 to include the medical finding 236, then the medical finding 236 is inserted (at block 708) into a field 206 (FIG. 2a) in the patient report 108. If (at block 710) the user settings 300 indicate in indicator 306 to include the BPR 232, then the BPR 232 inserted (at block 712) into a field 210 (FIG. 2a) in the patient report 108. If (at block 714) the user settings 300 indicate in indicator 308 to include the reference to publication for the BPR 234, then the reference 234 is inserted (at block 716) into a field 212 (FIG. 2a) in the patient report 108. The updated patient report 108 with the added information from the alert panel 230 is sent (at block 718) to the server 102 to process.

With the embodiment of operations of FIG. 7, user settings 300 for a particular user or radiologist are used to automatically determine the generated outcomes 128, 132 to insert into the patient report. This allows different radiologists to control what type of outcome information is inserted into a patient report 108 based on their preferences.

In one embodiment, the outcomes alert 136 may be encoded with the user settings 300 to be used by the report user interface 200 to control what outcomes 128, 132 are inserted into the patient report 108. In an alternative embodiment, the report user interfaced 200 may maintain a copy of the user settings 300 to use to control the outcomes 128, 132 inserted into the patient report.

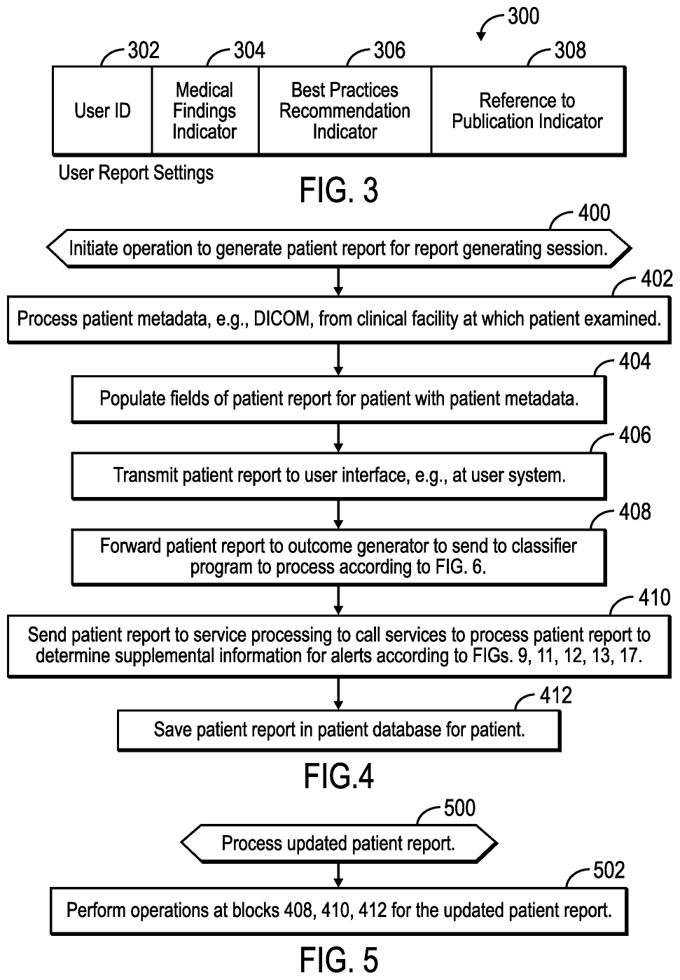

FIG. 8 illustrates an embodiment of a supplemental information tracking instance 800 stored in the patient database 120 to keep track of an extent to which a user has inserted content for supplemental information indicating deficiencies with the patient report. The supplemental information tracking instances 800 may be used to track variations in reimbursements when users users/radiologists adhered to the changes suggested by the supplemental information or did not adhere to the suggestions. A supplemental information tracking instance may indicate a patient identifier (ID) 802, a patient report identifier (ID) 804 of a patient report 108, a supplemental information alert 806 indicating a type of the supplemental information to render in an alert 240, 242 (FIG. 2d), such as missing sections, attestations to add, standardization of inhomogeneous descriptions, etc.; and an indicator 808 whether content for supplemental information was added to the patient report 108 or has not yet been added, i.e., acted upon.

FIG. 9 illustrates an embodiment of operations performed by the service processing 129 components to generate supplemental information 140a, 140b for alerts in the report user interface 200. Upon the orchestrator 126 receiving (at block 900) an initial 114 or updated 118 patient report, for each service 138a, 138b relevant to the patient report, the orchestrator 126 performs a loop of operations at blocks 904 through 908 for each service i relevant to the patient report 114, 118. At block 906, the called service i processes the patient report 114, 118 to determine whether to provide supplemental information 140a, 140b for the patient report. If (at block 908) the service i determines supplemental information 140a, 140b should be provided and if (at block 910) there is no tracking instance 800 in the patient database 120 for the determined supplemental information 806 and patient report 804, the orchestrator 126 adds (at block 912) tracking information 800 for each instance of supplemental information for the patient report 114, 118 indicating the supplemental information 806 and indicating in field 808 that content for the supplemental information 806 has not yet been inserted. From block 912 or if (at block 910) there is a already tracking instance 800 for the patient, report and supplemental information, indication is made (at block 914) in field 808 that content for the supplemental information was not inserted into the patient report 804. A supplemental information alert 144 is generated (at block 916) for the determined instance of supplemental information 140a, 140b, which is then transmitted to the report user interface 200d to render the alert 240, 242 (FIG. 2d). Control then proceeds (at block 918) back to block 904 until all services called.

With the embodiment of FIG. 9, multiple services 138a, 138b may be simultaneously or serially called to process the received patient report to determine supplemental information to provide in an alert in the report user interface 200d to alert of the deficiency in the patent report 108.

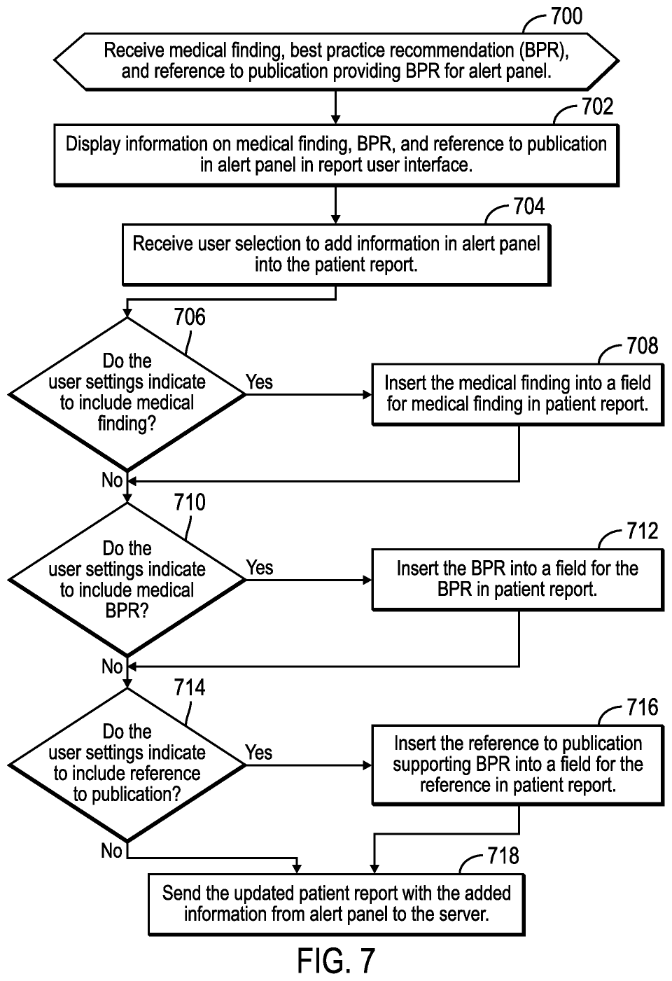

FIG. 10 illustrates an embodiment of operations performed by the report user interface 200d to render a supplemental information alert 144 from the server 102. Upon receiving (at block 1000) supplemental information alert 144, the report user interface 200d generates a supplemental information alert 240, 242 in the user interface 200d with user interface controls to enable the user to enter content for the supplemental information into the patient report 108. Upon receiving (at block 1004) in the report user interface 200d user selection to insert content for the supplemental information 140a, 140b into the patient report, such as attestations of patient examination descriptions, standardized terms for inhomogeneous content, missing sections, etc., the report user interface 200d inserts (at block 1006) content into the patient report to produce an updated patient report 108. The updated patient report 108 is transmitted (at block 1008) to the server 102 to perform additional processing at the outcome generator 124 and service processing 129. The report user interface 200d transmits (at block 1010) indication that the user inserted content for supplemental information to the server 102 to update the indication 808 in the supplemental tracking information instance 800 to indicate the content for the supplemental information was added in field 808.

With the described embodiments of FIG. 10, the user may update the patient report 118 with content for the supplemental information rendered in the alert panels 240, 242 to correct discrepancies determined by the services 138a, 138b.

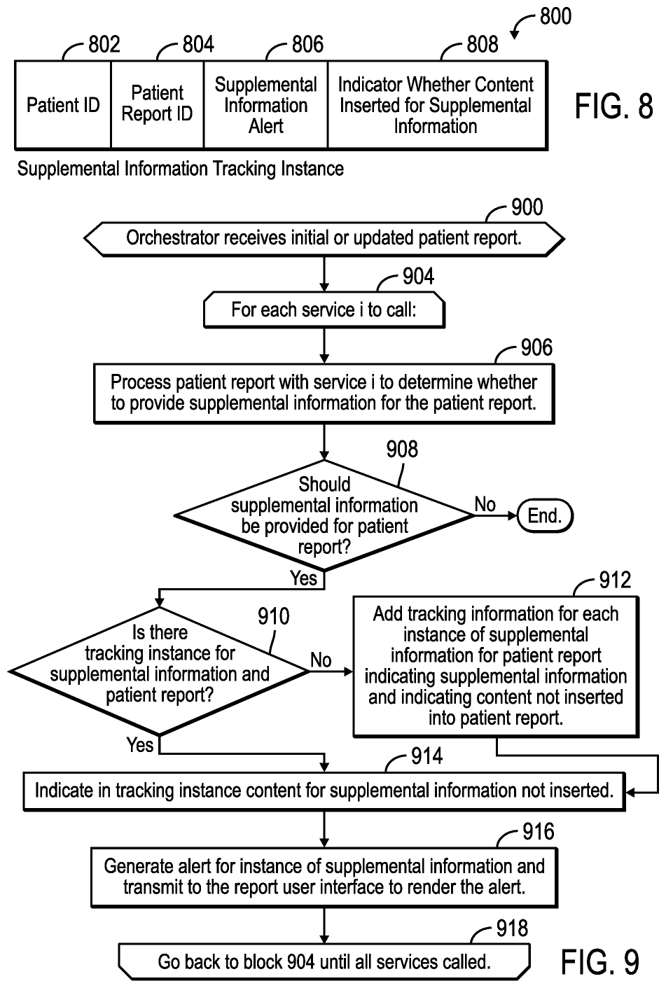

FIG. 11 illustrates an embodiment of operations performed by a service 138b to process a patient report 114, 118 to determine inhomogeneous content to standardize, such as medical descriptions that are not in a recognized master data set, domain or taxonomy for an organization deploying the service 138b. Upon calling (at block 1100) a service 138b that addresses undefined acronyms, the service 138b uses (at block 1102) natural language processing (NLP), or other programming techniques, to determine inhomogeneous content not matching homogeneous/standardized content defined in master data set for a domain or taxonomy. The service 138b generates, using NLP, (at block 1104) an interpretation of the inhomogeneous content, such as in the form of tags classified from features extracted from the inhomogeneous content and its context in the patient report 114, 118. 108. The service 138b determines (at block 1106) standardized/homogeneous content matching the interpretation of the inhomogeneous content, such as having same tags or having interpretation codes matching within a specified confidence level. The alert generator 142 generates a supplemental information alert 144, including the inhomogeneous content and the standardized content, to send to the user system 100 to render in the report user interface 200. The report user interface 200 may render an alert panel 110 to render information on the inhomogeneous content and the standardized content included in the transmitted alert 144 to enable the user to select to substitute the inhomogeneous content with the standardized content in the patient report 108. Indication of substituting inhomogeneous content with standardized content is transmitted to the server 102 to update the tracking information 800 for the standardized content alert to indicate the supplemental information is added, as described in FIGS. 9 and 10.

With the embodiment of FIG. 11, the service 138b uses natural language processing to interpret and translate inhomogeneous content to determine standardized content from standardized, homogeneous sets defined by an organization, such as an organization or domain master data set. By standardizing content in the patient report, the classifier program 122 and other algorithms can be trained to interpret standardized content, regardless of the source of the content which may originate from different clinical systems using inhomogeneous terms. Use of standardized content recognized by the billing and reimbursement processor will facilitate greater rates of reimbursement based on the patient reports.

An example of inhomogeneous content in the patient report 108 may comprise “CT Chest WWO IVC”. The service 138b may translate this non-standardized term format into a standardized format such as “CT CHEST WITH AND WITHOUT IV CONTRAST”.

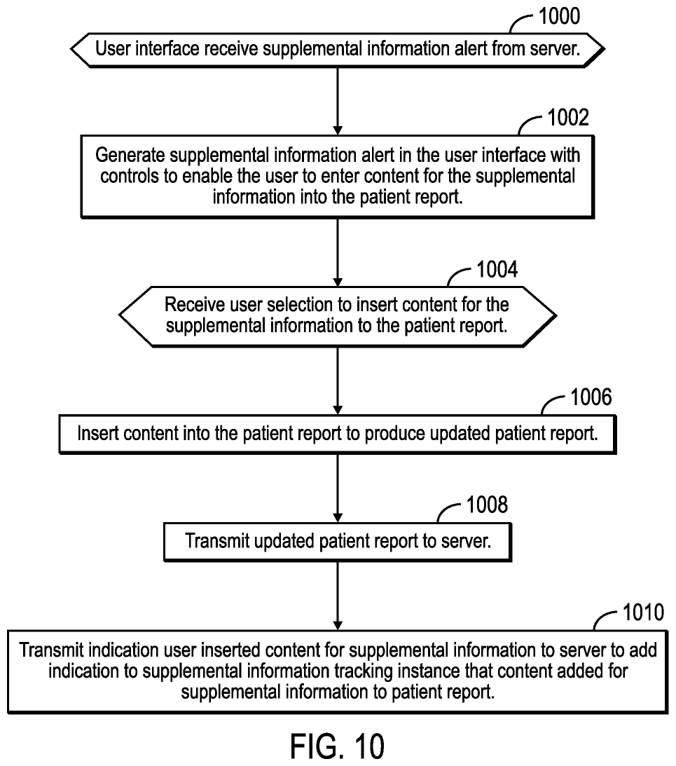

FIG. 12 illustrates an embodiment of operations performed by a service 138b to determining patient information in the patient report 114, 118 that is missing or has discrepancies. Upon calling (at block 1200) a service 138b that addresses discrepancies or missing patient information, the service 138b processes (at block 1202) the patient report 114, 118 to determine missing patient data. The service 138b may further process (at block 1204) the patient report 114, 118 to determine discrepancies between information in the patient report 114, 118 and the patient medical records in the patient database 120. If there is missing patient data or discrepancies, then the alert generator 142 generates (at block 1206) a supplemental information alert 144 including indicating patient metadata in patient report missing and/or having discrepancies and send to user interface to render in the report user interface 200 to prompt the user to enter corrections for the patient metadata indicated as missing or having discrepancies. The report user interface 200 may then transmit indication to the server 102 to update the tracking information 800 for the alert to indicate missing or corrected patient information added to the report 114, 118, as described in FIGS. 9 and 10.

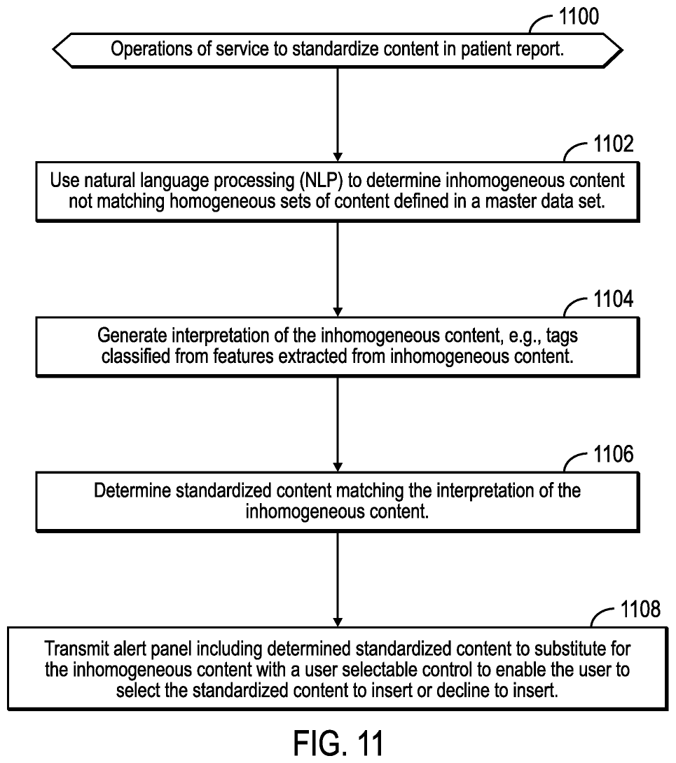

FIG. 13 illustrates an embodiment of operations performed by a service 138b to determining missing sections from the patient report 108. Upon calling (at block 1300) a service 138b that determines missing sections in the patient report 108, the service 138b, using natural language processing and/or term lookup, processes (at block 1302) the patient report 114, 118 to determine a missing predefined section. The alert generator 142 generates (at block 1304) supplemental information alert 144 having information on missing sections to send to the report user interface 200d (FIG. 2d) to render a missing section alert 240 to prompt the user to enter the missing section, such as a “History” section in the example of alert 240 in FIG. 2d.

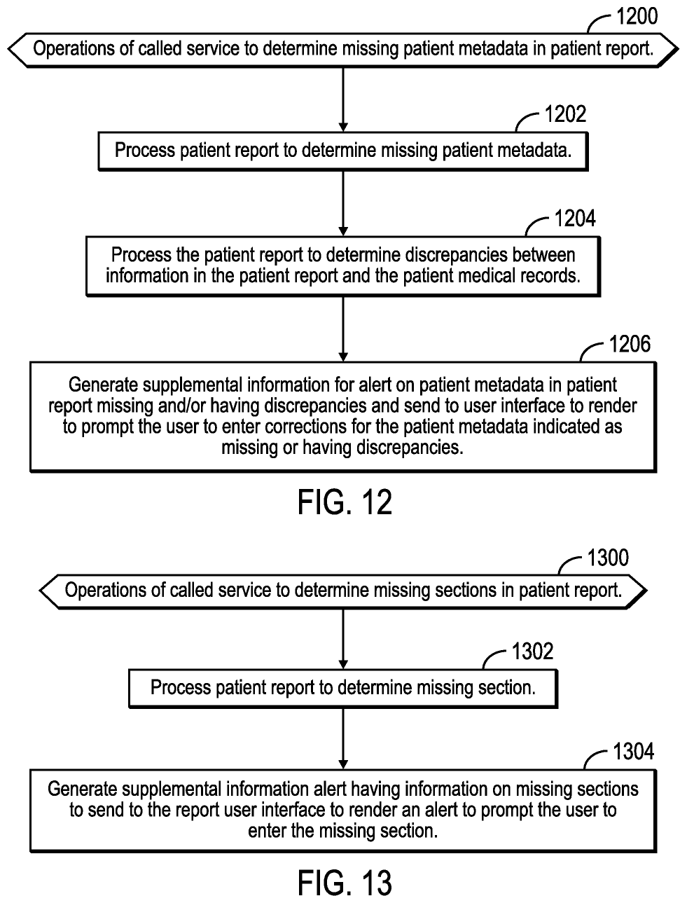

FIGS. 14-16 provide embodiments of an attestation service to prompt the user to add an attestation for a patient examination mentioned in the patient report 108 that provides explanatory information on the examination, such as doses used, machine settings, etc., to optimize reimbursements. FIG. 14 provides an embodiment of an attestation service 1400, such as one of the services 138a, 138b, that includes attestation associations 1402 to associate attestations providing explanations and information for a patient examination type with parameters. Different attestations may be provided in the associations 1402 for patient examination types having different testing and administration parameters. A natural language processor (NLP) 1404 may process the patient report 1406, such as patient reports 114, 118, to determine mentioned patient examinations and to determine if there is an associated attestation already included in the patient report 1406. Alternatively, the component 1404 may use a look-up table to search for terms for a patient examination or the attestation to determine if they are already included in the patient report 1406. The natural language processor 1404 will determine an attestation 1408 to present to the user to add to the patient report 1406. Further, an attestation tracking instance 1410, such as tracking information 800, may be added for the attestation being presented to the report user interface 200d (FIG. 2d) to present to the user in an attestation alert 242. In the example of FIG. 2d, the attestation 242 provides details on radiation dose lowering technique used for the imaging performed.

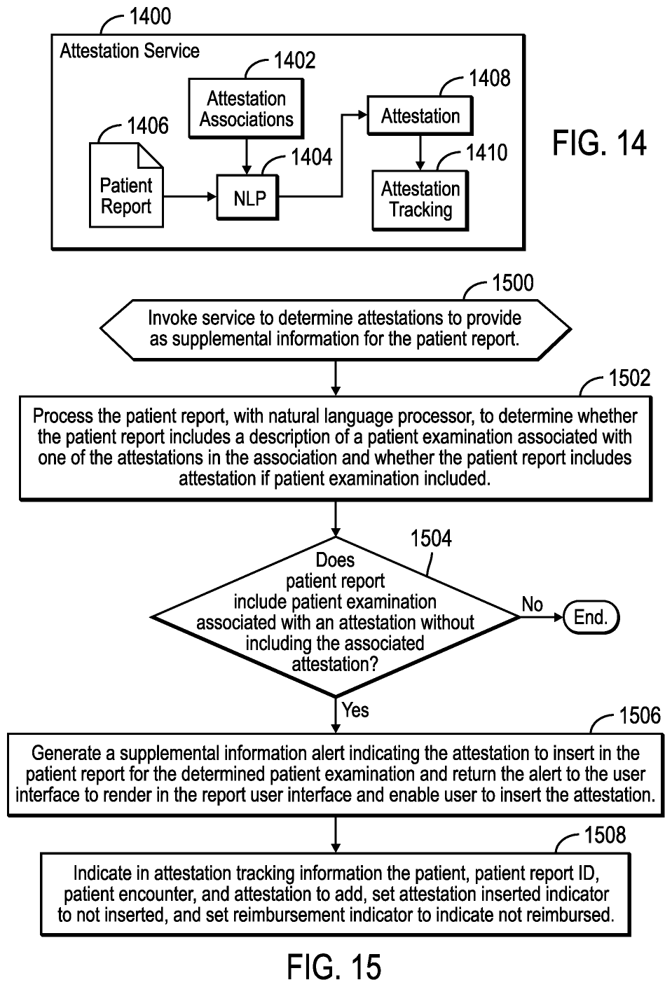

FIG. 15 illustrates an embodiment of operations performed by the attestation service 1400 to determine attestations to provide as supplemental information. Upon invoking (at block 1500) the attestation service 1400, the NLP 1404 processes (at block 1502) the patient report 1406, to determine whether the patient report 1406 includes a description of a patient encounter or examination associated with one of the attestations in the association 1402 and whether the patient report 1406 includes the attestation if the defined patient examination/encounter is included in the report 1406. If (at block 1504) the patient report 1406 includes a patient examination associated with an attestation not included in the report 1406, then the alert generator 142 generates (at block 1506) a supplemental information alert 144 indicating the attestation 1408 to insert in the patient report 1406 for the determined patient examination and returns the attestation alert 144 to the report user interface 200d (FIG. 2d) to render in the report user interface 200d and enable the user to insert the rendered attestation. Attestation tracking information 1410 is added (at block 1508) to the patient database 120 to indicate he patient, patient report ID, patient examination type and parameters, and attestation to add. An inserted indicator is set to indicate the attestation has not been inserted, and set reimbursement indicator to indicate not reimbursed. If (at block 1504) the attestation associated with the patient examination is already included in the patient report 114, 118, then control ends. The report user interface 200d may transmit indication to the server 102 to update the attestation tracking information 1410 for the alert to indicate the attestation was added to the patient report 1406, as described in FIGS. 9 and 10.

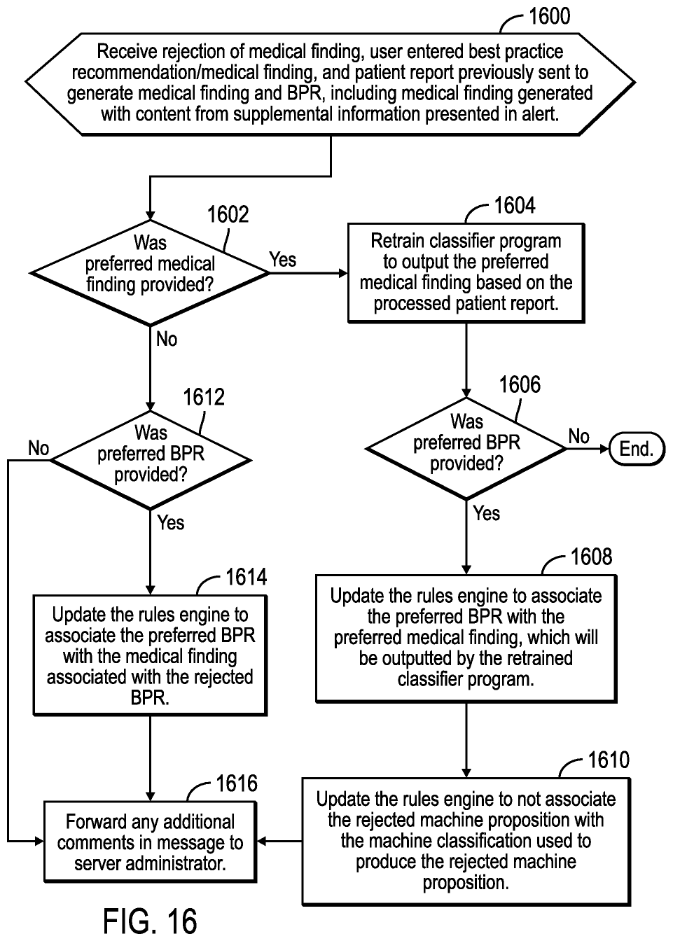

FIG. 16 illustrates an embodiment of operations performed by the retraining program 137 to process a message sent from the report user interface 200 when the user rejects a medical finding and/or best practice recommendation from the outcome generator 124, including a medical finding generated with content from supplemental information presented in an alert. The message from the report user interface 200 may include information on the rejected medical finding and medical best practice recommendation, a user indicated preferred medical finding/best practice recommendation, the content in the patient report 114, 115 that was processed to generate the medical best practice recommendation 132 and medical finding 128, and any other feedback,

Upon receiving (at block 1600) the message, if (at block 1602) a preferred medical finding was provided, then the retraining program 137 trains the classifier program 122 (at block 1604) to output the user preferred medical finding as the machine classification based on the processed patient report that resulted in the previously sent rejected medical finding. If (at block 1606) a preferred medical best practice recommendation was provided when rejecting the sent medical BPR 132, then the rules engine 130 may be updated (at block 1608) to output the preferred medical best practice recommendation for the preferred medical finding, which the classifier program 122 has been retrained to produce. The rules engine 130 may further be updated (at block 1610) to not associate the rejected medical best practice recommendation that was previously found to be associated with the medical finding used to produce the rejected medical best practice recommendation, so as not to produce again the medical best practice recommendation that the user rejected given the current patient report content rendered in the user interface 200.

If (at block 1602) a preferred medical finding was not provided and if (at block 1612) a preferred medical best practice recommendation was provided, then the rules engine 130 is updated (at block 1614), subject to organizational approval, to output the preferred medical best practice recommendation for the medical finding used to produce the rejected medical best practice recommendation, so that the user suggested preferred proposition will be produced instead of the user rejected machine proposition for that machine classifier. From the no branch of block 1612, block 1614 or 1610, any additional comments provided with the message are forward to a server administrator to consider for improving the performance of the classifier program 122 and/or rules engine 130. Further, an administrator approval may be needed before updating the rules engine in blocks 1614, 1608, 1610.

The embodiment of FIG. 16 provides improved computer technology for retraining the classifier program 122 and/or rules engine 130 to produce user preferred output when the user rejects a medical best practice recommendation previously sent to the report user interface, which is based on a previous patient report and medical finding generated with content from supplemental information presented in an alert. This allows for continual improvement of the medical findings and medical best practice recommendations produced for a patient report as the patient report is modified with content for supplemental information 140a, 140b produced by services 138a, 138b continually processing reports as they are modified.

The present invention may be a system, a method, and/or a computer program product. The computer program product may include a computer readable storage medium (or media) having computer readable program instructions thereon for causing a processor to carry out aspects of the present invention.

The computer program product comprises a computer readable storage medium implemented using standard programming and/or engineering techniques to produce software, firmware, hardware, or any combination thereof. The described operations may be implemented as code or logic maintained in a “computer readable storage medium”. The term “code” and “program code” as used herein refers to software program code, hardware logic, firmware, microcode, etc. The computer readable storage medium, as that term is used herein, includes a tangible element, including at least one of electronic circuitry, storage materials, a casing, a housing, a coating, hardware, and other suitable materials. A computer readable storage medium may comprise, but is not limited to, a magnetic storage medium (e.g., hard disk drives, floppy disks, tape, etc.), optical storage (CD-ROMs, DVDs, optical disks, etc.), volatile and non-volatile memory devices (e.g., EEPROMs, ROMs, PROMs, RAMs, DRAMs, SRAMs, Flash Memory, firmware, programmable logic, etc.), Solid State Devices (SSD), computer encoded and readable punch cards, etc. The computer readable storage medium may further comprise a hardware device implementing firmware, microcode, etc., such as in an integrated circuit chip, a programmable logic device, a Programmable Gate Array (PGA), field-programmable gate array (FPGA), Application Specific Integrated Circuit (ASIC), etc. A computer readable storage medium is not comprised solely of transmission signals and includes physical and tangible components. Those skilled in the art will recognize that many modifications may be made to this configuration without departing from the scope of the present invention, and that the article of manufacture may comprise suitable information bearing medium known in the art.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

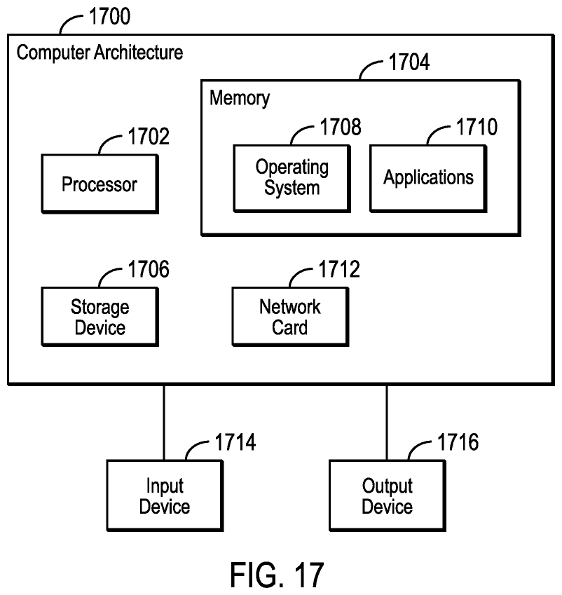

The computational components of FIG. 1, including the computer device 100, the server 102, and clinical facility 106, may be implemented in one or more computer systems, having a computer architecture as shown in FIG. 17, and including a processor 1702 (e.g., one or more microprocessors and cores), a memory 1704 (e.g., a volatile memory device), and storage 1706 (e.g., a non-volatile storage, such as magnetic disk drives, solid state devices (SSDs), optical disk drives, a tape drive, etc.). The storage 1706 may comprise an internal storage device or an attached or network accessible storage. Programs, including an operating system 1708 and applications 1710 stored in the storage 1706 are loaded into the memory 1704 and executed by the processor 1702. The applications 1710 may include the report user interface 200, patient report generator 112, client interface 116, classifier program 122, rules engine 130, outcome alert generator 134, retraining program 137, alert generator 142 and other program components described above. The architecture 1700 further includes a network card 1712 to enable communication with the network 16. An input device 1017 is used to provide user input to the processor 1702, and may include a keyboard, mouse, pen-stylus, microphone, touch sensitive display screen, or any other activation or input mechanism known in the art. An output device 1716, such as a display monitor, printer, storage, etc., is capable of rendering information transmitted from a graphics card or other component. The output device 1716 may render the GUIs described with respect to figures and the input device 1714 may be used to interact with the graphical controls and elements in the GUIs described above. The architecture 1700 may be implemented in any number of computing devices, such as a server, mainframe, desktop computer, laptop computer, hand held computer, tablet computer, personal digital assistant (PDA), telephony device, cell phone, etc.

The terms “an embodiment”, “embodiment”, “embodiments”, “the embodiment”, “the embodiments”, “one or more embodiments”, “some embodiments”, and “one embodiment” mean “one or more (but not all) embodiments of the present invention(s)” unless expressly specified otherwise.

The terms “including”, “comprising”, “having” and variations thereof mean “including but not limited to”, unless expressly specified otherwise.

The enumerated listing of items does not imply that any or all of the items are mutually exclusive, unless expressly specified otherwise.

The terms “a”, “an” and “the” mean “one or more”, unless expressly specified otherwise.

Devices that are in communication with each other need not be in continuous communication with each other, unless expressly specified otherwise. In addition, devices that are in communication with each other may communicate directly or indirectly through one or more intermediaries.

A description of an embodiment with several components in communication with each other does not imply that all such components are required. On the contrary a variety of optional components are described to illustrate the wide variety of possible embodiments of the present invention.

When a single device or article is described herein, it will be readily apparent that more than one device/article (whether or not they cooperate) may be used in place of a single device/article. Similarly, where more than one device or article is described herein (whether or not they cooperate), it will be readily apparent that a single device/article may be used in place of the more than one device or article or a different number of devices/articles may be used instead of the shown number of devices or programs. The functionality and/or the features of a device may be alternatively embodied by one or more other devices which are not explicitly described as having such functionality/features. Thus, other embodiments of the present invention need not include the device itself.

The foregoing description of various embodiments of the invention has been presented for the purposes of illustration and description. It is not intended to be exhaustive or to limit the invention to the precise form disclosed. Many modifications and variations are possible in light of the above teaching. It is intended that the scope of the invention be limited not by this detailed description, but rather by the claims appended hereto. The above specification, examples and data provide a complete description of the manufacture and use of the composition of the invention. Since many embodiments of the invention can be made without departing from the spirit and scope of the invention, the invention resides in the claims herein after appended.

1-45. (canceled)

46. A computer program product for updating a patient report rendered in a user interface, the computer program product comprising a computer readable storage medium having computer readable program code embodied therein that is executable to perform operations, the operations comprising:
processing the patient report to determine supplemental information for the patient report, wherein the patient report has information on a patient encounter at a medical clinic;
transmitting the supplemental information to the user interface to render in the user interface;
receiving a first updated patient report including content for the supplemental information a user of the user interface selected to insert to the patient report;
processing, by a classifier program, the first updated patient report including the content for the supplemental information to classify into a medical finding based on content in the patient report and the content for the supplemental information;
determining a medical best practice recommendation associated with the medical finding outputted from the classifier program;
transmitting the medical finding and the medical best practice recommendation to the user interface to render in the user interface; and
receiving a second updated patient report including at least one of the medical finding and the medical best practice recommendation the user selected to include into the patient report.

47. The computer program product of claim 46, wherein the operations further comprise:
determining whether the medical best practice recommendation and the medical finding differ from a medical best practice recommendation and medical finding indicated in the first updated patient report, wherein the determined medical finding and the medical best practice recommendation are only transmitted to the user interface in response to determining that the medical best practice recommendation and the medical finding differ from a medical best practice recommendation and medical finding indicated in the first updated patient report.

48. The computer program product of claim 46, wherein the user interface renders the medical finding and the medical best practice recommendation in an alert panel in the user interface.

49. The computer program product of claim 46, wherein the processing the patient report to determine the supplemental information comprises:
processing the patient report to determine inhomogeneous content;
generating an interpretation of the inhomogeneous content; and
determining standardized homogeneous content matching the interpretation of the inhomogeneous content, wherein the supplemental information provided to the user interface and the content inserted in the patient report comprises the determined standardized homogeneous content.

50. The computer program product of claim 46, wherein the operations further comprise:
populating fields of the patient report with patient metadata received from a clinical facility at which the patient was examined, wherein the processing the patient report to determine the supplemental information comprises processing the patient report to determine patient metadata in the patient report missing and/or having discrepancies that has a strong predictive quality for the medical finding, wherein the supplemental information comprises information on the patient metadata missing or having discrepancies,
wherein the receiving the content for the supplemental information comprises user input for the determined patient metadata inserted into the patient report, wherein the first updated patient report includes user entered corrections to the determined patient metadata.

51. The computer program product of claim 46, wherein the operations further comprise:
calling a plurality of services to each perform the operations of processing the patient report to determine the supplemental information and transmitting the supplemental information to the user interface, wherein different of the services produce different types of supplemental information to transmit to the user interface to render in the user interface, and wherein the received first updated report includes multiple instances of content for the different types of supplemental information, and wherein the first updated report with the content for the different types of supplemental information are provided to the classifier program to determine the medical finding.

52. The computer program product of claim 51, wherein the operations further comprise:
receiving an updated patient report including user selected modifications in response to the user modifying a currently rendered patient report in the user interface, wherein the currently rendered patient report comprises one of the patient report, the first updated patient report, the second updated patient report, and another version of the patient report currently rendered in the user interface; and
calling the plurality of services to each perform the operations of processing the updated patient report to determine the supplemental information and transmitting the supplemental information to the user interface.

53. The computer program product of claim 46, wherein the processing the patient report to determine the supplemental information comprises:
determining whether the patient report includes all required sections; and
determining a missing section in response to determining that the patient report does not include all the required sections, wherein the supplemental information provided to the user interface comprises information on the determined missing section and the content inserted in the patient report comprises user input for the missing section.

54. The computer program product of claim 46, wherein an additional instance of the operations of processing the patient report to determine the supplemental information and transmitting the supplemental information to the user interface are performed in response to receiving an updated patient report from the user interface.

55. The computer program product of claim 46, wherein an additional instance of the operations of processing the patient report to determine the supplemental information and transmitting the supplemental information to the user interface are continuously performed at time intervals.

56. The computer program product of claim 46, wherein the operations further comprise:
tracking instances in which supplemental information is transmitted to user interfaces for users;
indicating in the tracked instances whether users included content for the supplemental information in an updated patient report; and
processing the tracked instances and indication whether users included content for the supplemental information to analyze how often patient reports are updated with content for supplemental information to improve the patient report.

57. The computer program product of claim 46, wherein the operations further comprise:
determining reimbursements for a first set of patient reports for which content for the supplemental information was not included in an updated patient reports;
determining reimbursements for a second set of patient reports for which content for supplemental information was inserted into the patient reports; and
determining an extent to which inserting the content for the supplemental information results in improved reimbursements for the patient reports.

58. A system for updating a patient report rendered in a user interface, comprising:
a processor; and
a computer readable storage medium having computer readable program code that when executed by the processor performs operations, the operations comprising:
processing the patient report to determine supplemental information for the patient report, wherein the patient report has information on a patient encounter at a medical clinic;
transmitting the supplemental information to the user interface to render in the user interface;
receiving a first updated patient report including content for the supplemental information a user of the user interface selected to insert to the patient report;
processing, by a classifier program the first updated patient report including the content for the supplemental information to classify into a medical finding based on content in the patient report and the content for the supplemental information;
determining a medical best practice recommendation associated with the medical finding outputted from the classifier program;
transmitting the medical finding and the medical best practice recommendation to the user interface to render in the user interface; and
receiving a second updated patient report including at least one of the medical finding and the medical best practice recommendation the user selected to include into the patient report.

59-60. (canceled)

61. The system of claim 58, wherein the processing the patient report to determine the supplemental information comprises:
processing the patient report to determine inhomogeneous content;
generating an interpretation of the inhomogeneous content; and
determining standardized homogeneous content matching the interpretation of the inhomogeneous content, wherein the supplemental information provided to the user interface and the content inserted in the patient report comprises the determined standardized homogeneous content.

62. The system of claim 58, wherein the operations further comprise:
populating fields of the patient report with patient metadata received from a clinical facility at which the patient was examined, wherein the processing the patient report to determine the supplemental information comprises processing the patient report to determine patient metadata in the patient report missing and/or having discrepancies that has a strong predictive quality for the medical finding, wherein the supplemental information comprises information on the patient metadata missing or having discrepancies,
wherein the receiving the content for the supplemental information comprises user input for the determined patient metadata inserted into the patient report, wherein the first updated patient report includes user entered corrections to the determined patient metadata.

63. The system of claim 58, wherein the operations further comprise:
calling a plurality of services to each perform the operations of processing the patient report to determine the supplemental information and transmitting the supplemental information to the user interface, wherein different of the services produce different types of supplemental information to transmit to the user interface to render in the user interface, and wherein the received first updated report includes multiple instances of content for the different types of supplemental information, and wherein the first updated report with the content for the different types of supplemental information are provided to the classifier program to determine the medical finding.

64-69. (canceled)

70. A method for updating a patient report rendered in a computer user interface, comprising:
processing the patient report to determine supplemental information for the patient report, wherein the patient report has information on a patient encounter at a medical clinic;
transmitting the supplemental information to the user interface to render in the user interface;
receiving a first updated patient report including content for the supplemental information a user of the user interface selected to insert to the patient report;
processing, by a classifier program the first updated patient report including the content for the supplemental information to classify into a medical finding based on content in the patient report and the content for the supplemental information;
determining a medical best practice recommendation associated with the medical finding outputted from the classifier program;
transmitting the medical finding and the medical best practice recommendation to the user interface to render in the user interface; and
receiving a second updated patient report including at least one of the medical finding and the medical best practice recommendation the user selected to include into the patient report.

71-72. (canceled)

73. The method of claim 70, wherein the processing the patient report to determine the supplemental information comprises:
processing the patient report to determine inhomogeneous content;
generating an interpretation of the inhomogeneous content; and
determining standardized homogeneous content matching the interpretation of the inhomogeneous content, wherein the supplemental information provided to the user interface and the content inserted in the patient report comprises the determined standardized homogeneous content.

74. The method of claim 70, further comprising:
populating fields of the patient report with patient metadata received from a clinical facility at which the patient was examined, wherein the processing the patient report to determine the supplemental information comprises processing the patient report to determine patient metadata in the patient report missing and/or having discrepancies that has a strong predictive quality for the medical finding, wherein the supplemental information comprises information on the patient metadata missing or having discrepancies,
wherein the receiving the content for the supplemental information comprises user input for the determined patient metadata inserted into the patient report, wherein the first updated patient report includes user entered corrections to the determined patient metadata.

75. The method of claim 70, further comprising:
calling a plurality of services to each perform the operations of processing the patient report to determine the supplemental information and transmitting the supplemental information to the user interface, wherein different of the services produce different types of supplemental information to transmit to the user interface to render in the user interface, and wherein the received first updated report includes multiple instances of content for the different types of supplemental information, and wherein the first updated report with the content for the different types of supplemental information are provided to the classifier program to determine the medical finding.

76-81. (canceled)
